(19) United States
(12) Patent Application Publication
(10) Pub. No.: US 2010/0226894 A1
(43) Pub. Date: Sep. 9, 2010

(54) Mobilization of a complete cells mixture with embryonic like stem cells from the peripheral blood

(76) Inventors: Alex Wah Hin Yeung, Newport Beach, CA (US); Kimberly Jane Yeung, Newport Beach, CA (US)

Correspondence Address: YEUNG, Alex Wah Hin, 2 Narbonne, Newport Beach, CA, 92660, US

(21) Appl. No.: 12/660,137

(22) Filed: Feb. 22, 2010

Related U.S. Application Data
(60) Provisional application No. 61/209,448, filed on Mar. 9, 2009.

(51) Int. Cl. A61K 35/14; A61P 19/04

(52) U.S. Cl. 424/93.7

(57) ABSTRACT

Disclosed is a method to recover a mixture cell population consisting of a complete profile of progenitor cells including embryonic like stem cells (a “complete progenitor cell mixture” or CPM) from the peripheral blood of an individual by administering to that individual a combination of at least one or more from the category of growth factors and hormones combining with at least one or more from the category of cell fusion inhibitor compounds, and then recovering peripheral blood progenitor cells from said individual. The hormones and growth factors group should include hGH (human growth hormone) with or without erythropoietin (EPO), but specifically without either G-CSF or GM-CSF. The cell fusion inhibitor group should include a CXCR4 antagonist. Also disclosed is a method for using or preserving such a complete progenitor cells mixture (CPM) for the treatment of diseases.

### BACKGROUND OF THE INVENTION

Studies have shown that bone marrow derived progenitor cells are indeed promising in the treatment of diseased myocardium. The hematopoietic stem cell (HPC as CD34+ or AC133+), endothelial progenitor cell (EPC) and bone marrow derived mesenchymal stem cell or multipotent stromal cell (MSC) have all been shown to have the capability to either improve the perfusion and new vascular development within the infarct (Husnain 2005) or can, most often only in animal studies with limited actual cell numbers, actually differentiate into cardiomyocytes (Shake 2002). The mixture of progenitor cells that are being used, either from the bone marrow or from the peripheral blood mobilization, usually consist of either isolated or cultured EPC and MSC. The end result of which usually translates to improvement of the different sets of cardiac function assessment. Even though it was generally agreed that the paracrine effect was the most significant result of such stem cell infusion or injection, the hope had always been, and will be, that some of the EPC and MSC, if given enough numbers and be attracted to the right milieu environment, be able to differentiate into useful cardiomyocytes. Most of the studies so far, regardless whether these are done with animals or phase I clinical trials in humans, have been carried out using bone marrow cells or mononuclear cells (MNC). Interpretation of these results are also made more difficult by a large varieties of how these bone marrow progenitors cells are being harvested, selected and sometimes pre-cultured. Recently studies comparing the use of mobilization and collection of these progenitors (MNC fraction) from peripheral blood circulation show that the results are more or less equivalent (Assumus 2002). Studies in other diseases such as stroke, diabetic neuropathy etc., although not as advanced as in the cardiac model, follow more or less the same trend of guarded optimism.

Peripheral blood derived progenitors (PBSC) usually are mobilized by growth factors acting on the bone marrow. The use of G CSF and GM CSF as mobilization agents for autologous stem cell support after high dose chemotherapy dated back nearly twenty years during the time when such therapy was popular with lymphomas and chemosensitive solid tumors such as late stage breast cancer (Yeung 1994). It was well known that a large number of HPC, with some EPC and a minimal amount of MSC, can be collected using a blood cell separator after 4 days of G CSF stimulation. There have been relatively few clinical studies using these progenitors without further ex vivo manipulation since most presumed, and rightly so, that the number of EPC or MSC collected will be very low with this methodology. There has been no credible report, before this invention, claiming that MSC or any associated stromal type progenitor cells directly from PBSC can contribute to engraftment or differentiation into functional specialized cell like the cardiomyocytes (Cashen 2007). This invention specifically will not employ any G-CSF or GM-CSF, growth factors either of which has been the choice in all PBSC preparations so far.

Recently very small embryonic like stem cell (VSEL) have been identified in a variety of organs (bone marrow and cord blood) and a small number of these can be found in the peripheral blood of experimental animals (Kucia 2008), with or without G CSF mobilization. The numbers are unusually low (8-10/ml of blood in human (Wojakowski 2009)) but they exhibit some markers characteristic for embryonic stem cells, epiblast stem cell and primordial germ cells. The function of these cells is unknown and their presence is usually limited in younger animals only. In this invention, we can show that, by either one or a combination of more than one hormones or growth factors, adult embryonic like stem cells (AELC) can be mobilized in reasonable and directly useable numbers into the peripheral blood, which then can be collected for use in the treatment of damaged or diseased tissues or organs.

EPO has been shown recently to enhance the expression of VEGF in cardiac as well as bone marrow endothelial cells, thereby increases the survival, proliferation and differentiation of EPC (Westenbrink 2007). EPC from bone marrow are mobilized in large numbers into the peripheral blood. In addition, VEGF expression by myocardial endothelial cells exert chemotactic effect on these circulating EPC, giving rise to a synergistic effect on cardiac endothelial repair. Nothing is known about its effect on adult embryonic like stem cells in the peripheral blood.

Human Growth Hormone (hGH), is a protein hormone secreted by the pituitary gland and it plays a key role in somatic growth through its metabolic effects affecting proteins, fat and carbohydrates. It has also been shown, with the help of G-CSF, to mobilize CD 34+ cells (Carlo-Stella 2004) in the peripheral blood and helps in the engraftment of hematopoietic stem cells in bone marrow transplant. It has not been shown to have any effects on EPC, MSC and other adult embryonic like stem cells.

Plerixafor, a macrocyclic compound antagonist of the alpha chemokine receptor CXCR4, was approved for HSC mobilization. The SDF-1/CXCL12/CXCR4 retention axis disruption by this agent in the bone marrow can release a whole host of progenitor cells without the necessity of priming. The result in HSC collection using G CSF and Plerixafor has been dramatic. A recent report using VEGF priming, instead of G CSF, and Plerixafor results in a differential mobilization of a large number of EPC and MSC (together with HSC) into the peripheral blood (Pitchford 2009). Nothing is known about its effect on adult embryonic like stem cells in the peripheral blood.

The methodology of peripheral blood collection of progenitor cells to be used for clinical trials has logistic and technical advantages over bone marrow preparations. The methodology by itself is minimally invasive, and can be preformed easily in an outpatient setting instead of in an operation theater or a clean room suite. The use of a closed and self cleansing automated system means that the product can be used either immediately or be easily transported for storage and cryopreservation for repeated use in the future without any manipulation.

An ideal stem cell treatment is still many years away, as there are still a number of unanswered questions of how to use embryonic stem cells or the newly discovered induced pluripotent stem cells by gene transfer. Even if they can exhibit the “perfect” phenotype in vitro, how these cells will migrate, engraft and grow synergistically in the microscopic milieu environment in disease tissues or organs is still in question. Any mishap of a few cells, during the long and complicated ex vivo manipulation, can lead to dire consequences. This is especially true when a recent report showed that a donor receiving embryonic stem cell transplant developed brain tumor (Amariglio, 2009). In addition, the cost of an individualized stem cell treatment, considering the need for induction, culture and all the safety issues that have to be built in, will likely be just beyond the reach of most patients.

A good and practical alternative is to perfect the way that we can collect sufficient “stem” or stem like cells from an autologous, or sometimes, allogeneic source. PBSC, with its long history of establishment in the treatment of lymphoma and multiple myeloma, is one of such alternatives. This invention brings the best mobilization method for four of these progenitor cells as a “Complete Progenitor Cells” (CPM) mixture. Adult embryonic like stem cell AELC will be the primary force behind rapid cell renewal and differentiation along the induced cell type (e.g. as cardiomyocytes). MSC will be responsible for cell survival as well as specific cell differentiation. EPC will be responsible for microvascular expansion in cooperation with the newly generated cells as well as for the expected hypertrophy of the survivable cells. HPC, with its monocyte fractions, together with MSC will be responsible for immune-suppression (reducing the need for HLA matching), minimizing inflammation and fibrosis, leading for rapid repair to set in without extensive scarring (the ultimate goal, e.g., in treating myocardial or other infarction). Since CXCR4 antagonist induced PSBC with the CPM mixture cells are equipped with the homing receptor CXCR4, most of these cells will be attracted and stay at the disease site.

This invention will set forth the different factors and criteria so that CPM can be collected from PBSC technique and then be used clinically in patients as a tool for regeneration medicine.

### SUMMARY OF THE INVENTION

Throughout the long history of using PBSC for the treatment of cancer, it has never been known to produce enough EPC or any MSC that can be used for regenerative medicine (Cashen 2007). PBSC has always been, up to now, an alternative to bone marrow transplantation for the treatment of lymphomas and multiple myeloma. Some PBSC trials established reasonable improvement in autoimmune diseases. Its use as a supply of stem cell replacement therapy in regenerative medicine was reasonably established for its overall paracrine action in cardiac muscle revascularization. No significant, if any, cardiomyocyte regeneration can be expected from such a treatment.

This invention is based on the hypothesis and then discovery that hGH is effective, apart from its know ability to increase CFU-GM and BFU-E in vitro, in uniquely providing a rapid cell renewal of MSC and adult embryonic like stem cells (AELC) that are inherently associated with pericyte niches throughout multiple organs in the human body. Such rapid cell renewal turnover, together with subsequent breakdown of fusion molecules by inhibitors, will release sufficient numbers of MSC and AELC into the peripheral blood. Another uniqueness of the invention is that it does not utilize any G-CSF or GM-CSF, which up to now, are the only effective mobilization agents for any type of stem cells in PBSC collection.

This invention also is based on the discovery that hGH is synergistic with EPO in providing the necessary stimulation of EPC during anabolic and body building growth process that is the natural bodily defense system after hypoxia and massive cell injury.

After sufficient multiple stem cells build up and peripheral cell release with hGH and EPO priming, additional mobilization is supplied by a cell adhesion inhibitor such as CXCR4 antagonist, yielding a rich CPM with CXCR4 expression.

This CPM can then be harvested by the usual PSBC collection technique and system well known to those familiar with the art.

CPM can then be used immediately or be cryogenically preserved for future treatment by the same individual or by any other individual with appropriate disease tissue or damaged organ.

### DETAILED DESCRIPTION OF THE INVENTIONS

In a first aspect, the invention uses a method of preparation of different population of peripheral blood cells that can be termed as a complete progenitor cell mixture (CPM), with HPC, EPC, MSC and adult embryonic like stem cells.

This preparation of CPM, with or without further ex vivo manipulation, will be able to use for treating diseases that are well known to those skill in the art.

This invention specifically, in contrast to all other forms of PBSC mobilization, does not employ the use of either G-CSF or GM-CSF for the mobilization of CPM.

The invention employs a method to generate CPM by administering to a donor a composition comprising of a hormone, preferably hGH, one of its derivatives or any factor inducing its release in an amount sufficient in said donor the number of cells in the CPM recovered by PBSC technique for treatment purposes.

The invention employs a method to generate CPM by administering to a donor a composition comprising another growth factor hormone, preferably EPO, one of its derivatives or any factor inducing its release in an amount sufficient in said donor the number of cells in the CPM recovered by PBSC technique for treatment purposes.

The invention employs a method to generate CPM by administering to a donor a composition comprising a cell adhesion inhibitor, preferably CXCR4 antagonist, one of its derivatives or any factor inducing its release in an amount sufficient in said donor the number of cells in the CPM recovered by PBSC technique for treatment purposes.

The invention employs the combination of hGH, EPO and a CXCR4 antagonist in an amount sufficient to generate CPM that can be recovered from the peripheral blood.

In another aspect, this invention identifies a unique mixture population of stem cells in the peripheral blood known as CPM with HPC, EPC, MSC and AELC with known marker characteristics demonstrated by the following assay.

CFU-HPC Assay: 5×10⁴ cells were added to Methocult medium for 11 days incubation before quantification by aspiration into a single cell suspension before cell count. Immunostaining will consist of CD115, CD34, CD45, VEGFR1, VEGFR2, VE-Cadherin and vWF.

CFU-EPC Assay: 5×10⁵ cells were added to EPC colony media with EGM-2 basal media+ supplements, additional VEGF, 16% FBS on fibronectin plates incubation for 7 days, and then media changed for another 14 days of incubation before enumeration of CFU-EPC colonies expressing CD34, VEGFR2, VE-Cadherin and vWF. The late outgrowth CFU-EPC will not express CD115, CD14 or CD45, and are therefore not a monocyte-macrophage lineage. Alternately EPC can be assessed by incubation of adherent cells after 7 days with 1,1′-dioctadecyl-3,3,3′, 3′-tetramethylindocarbocyanine-labelled acetylated LDL for 12 hours, fixed with 1% paraformaldehyde for 10 minutes and counterstained with florescein isothyiocyanate-labelled Griffonia simplicifolia lectin I, isolectin B4 and DAPI for nuclear staining. Cells double positive for Dil AcLDL and lectin staining will be considered EPC and counted.

CFU-MSC Assay: 5×10⁵ cells were added to Mesencult media with supplements for initially 7 days with a further 14 days of incubation after media change. Enumeration of CFU-F for MSC from plastic adherent cells will be able to show expression of CD29 and CD105, but will be negative for CD45 and CD34. Other markers such as CD73, CD90, CD146, CD200, NG2 and PDGRbeta may also be tested as an option. Testing also include expanded MSC colonies assessed for their ability to differentiate into cells types like adipocytes, osteoblasts and chondrocytes.

AELC Assay: Identified by membrane markers such as Sca-1+Lin-CD45− and also with immune-fluorescent protein staining of Oct4, SSEA4 and Nanog.

In another aspect, this invention identifies cells in the CPM exhibit CXCR4 expression by antibody and FACS assay.

In another embodiment of this invention, apheresis is used to isolate CPM from the peripheral blood using known blood cell separators (PBSC technique) that are now available in the commercial market. Examples are machines made by Haemonetics V50 blood separator, the Baxter CS 3000, the Fresenius AS 104 and the Fresenius AS TEC 104 and the Excel. By varying the separation method, apheresis can be adapted to isolate different cellular components from the peripheral blood. In the case of CPM, the methodology employs those that separate the mononuclear cell fraction.

In a further aspect of this invention, PBSC technique is able to generate CPM in a closed system by an outpatient facility. The final CPM product is cleansed and further concentrated by density gradient centrifugation in an automatic process and be available for immediate use or be cryogenically stored for later use. Cryogenic preservation methods are well known in the art. These cells can also be expanded ex vivo using known methods.

In another embodiment, cells in the CPM can further be enriched differentially by those based on surface antigens expressed by certain types of stem cells, e.g. using FACS so that the fractions of the different types of stem cells in the CPM can be altered. Alternatively, cells can be sorted by mixing with magnetic beads coated with monoclonal antibodies against a cell surface antigen characteristically expressed by stem cells. In summary, any known methodology in the art can be employed to further change the composition of the different cell types within the CPM repertoire.

In another embodiment, CPM from this invention relates to methods of autologous transplantation, meaning the transplantation of tissues or cells from a subject's own body rather than from a donor individual.

The present invention also relates to methods of allogeneic transplantation. Allogeneic transplantation is the transplantation of tissues or cells from a genetically non-identically individual of the same species.

In a further embodiment, this invention is to utilize CPM as autologous or allogeneic transplantation to treat human tissue or organ that has been damaged or is diseased. Such damage or disease may have occurred in a number of ways such as by an infarction, mechanical injury, hypoxia, infection by bacteria or virus, hemorrhage, or as a result of an inherited or genetic condition, chemotherapy or irradiation. Aging and aging related conditions, though not a disease, may also produce organ damage or degeneration that can be substantially reversed by this invention.

In another well known example of infarction injury that results in tissue necrosis, and more particularly, a myocardial infarction, this invention will provide the implanting of CPM cells by different methods so that the disease damage may be reduced by the autocrine, paracrine, cell regeneration and micro revascularization.

In a further embodiment, the use of CPM can help in the engraftment or regeneration of other specialized cell treatments, usually prepared ex vivo before implantation by the culturing of, but not exclusively limited to, embryonic stem cell (ESC), induced pluripotent stem cell (iPS), pluripotent cells extracted from cord blood, placenta, umbilical cord, skin, fat, sex organs etc. The use of CPM can be given before, during or after the implantation of these specialized cell treatments, and can be used both in the autologous or allogeneic settings.

The methods of the invention can be used to treat any organs and tissues in need thereof. Non limiting examples of tissues include bone, cartilage, striated, skeletal and cardiac muscle. Non-limiting examples of organs that can be treated by CPM in this invention include heart, liver, brain, pancreas, kidney, intestine, lung, eye, bladder, spinal cord and skin.

The number of CPM cells delivered to the site of damage, or disease can also vary depending on the severity, size of the damaged or diseased area. The usual range can vary from 1×10⁴ to 1×10⁸.

In another embodiment, the CPM cells can be included in any formulations that are suitable for administration either into the bloodstream or be directly introduced into the disease or damaged tissues or organs. A suitable format can easily be determined by a medical practitioner for each patient, tissue, and organ accordingly.

In a further embodiment, the CPM cells can be placed in acceptable carriers with formulations well known in the art (e.g. Remington's Pharmaceutical Sciences 16^{th} edition, Osol, A. Ed. 1980). The cells are preferably being formulated in a solution with a pH from 6.5 to 8.5. Excipients to bring the cell mixture solution to isotonicity can also be added, such as 4.5% mannitol, normal 0.9% saline or sodium phosphate. Other pharmaceutically acceptable agents can also be added to bring the solution to isotonicity, including, but not limited to dextrose, boric acid, sodium tartrate, propylene glycol or other inorganic or organic solutes.

### BIBLIOGRAPHY

Amariglio. “Donor derived brain tumor following neural stem cell transplantation in an ataxia telangiectasia patient.” PLoS Medicine 6, no. 2 (2009).

Assumus. “Transplantation of progenitor cells and regeneration enhancement in acut myocardial infarction.” Circulation 106 (2002): 3009-3017.

Carlo-Stella, C. “Use of recombinant human growth hormone plus recombinant human G-CSF for the mobilization and collection of CD34+ cells in poor mobilizers.” Blood 103 (2004): 3287-3295.

Cashen, A F. “Mobilizing stem cells from normal donors: is it possible to improve upon G-CSF?” Bone Marrow Transplantation 39 (2007): 577-588.

Husnain, K H. “Bone marrow stem cell transplantation for cardiac repair.” AJP Heart Circ Physiol 288 (2005): 2557-2567.

Kucia, M J. “Evidence that very small embryonic like stem cells are mobilized into peripheral blood.” Stem Cells 8 (2008): 2083-2092.

Pitchford, Simon. “Differential mobilization of subsets of progenitor cells from the bone marrow.” Cell Stem Cell 4 (2009): 62-72.

Shake, J G. “Mesenchymal stem cell implantation in a swine myocardial infarct model: engraftment and functional effects.” Ann Thorac Surg 73 (2002): 1919-1926.

Westenbrink, B D. “Erythropoietin improves cardiac function through endothelial progenitor cell and vascular endothelial growth factor mediated neovascularization.” European Heart Journal 28, no. 16 (2007): 2018-2027.

Wojakowski, W. “Mobilization of bone marrow derived Oct-4+ SSEA-4+ very small embryonic like stem cells in patients with acut myocardial infarction.” J Am Coll Cardiol 53 (2009): 1-9.

Yeung, Alex W. “Double cycle high dose chemotherapy with peripheral blood stem cell and hematopoietic growth factor support in patients with advanced solid tumor.” Cancer 73, no. 7 (1994): 1960-1970.

What is claimed is:

1. A method of preparation by means of mobilization and collection, from the peripheral blood of a subject, the different populations of peripheral blood cell in a complete progenitor cell mixture (CPM) of hematopoietic progenitor cells (HPC), endothelial progenitor cells (EPC), mesenchymal stem cells or multipotent stromal cells (MSC), adult embryonic like stem cells (AELC) by the administration of, but specifically without the use G-CSF or GM-CSF, a combination and effective amount of at least one or more from the category of growth factors and hormones with at least one or more from the category of cell fusion inhibitor compounds.

2. The method of claim 1, wherein the CPM preparation is for the treatment of damaged or diseased tissue or organ of human by the implanting of the CPM cells into the tissue or organ in need of treatment, whereby such an implantation ameliorates damage or disease of the tissue or organ.

3. The method of claim 1, wherein the hormone is human growth hormone (hGH), or one of its derivatives or any factor inducing its release.

4. The method of claim 1, wherein the growth factor is erythropoietin (EPO), or one of its derivatives or any factor inducing its release.

5. The method of claim 1, wherein the cell adhesion inhibitor can be from any known cellular, MMP related, chemokine ligand and receptor combinations etc., including but not limited to a CXCR4 antagonist.

6. The method of claim 1, wherein the subject is a human.

7. The method of claim 1, wherein the preparation of CPCM is done by apheresis or other methods of isolation known to the art from the peripheral blood of the subject.

8. The method of claim 7, wherein the donor is the subject to be treated.

9. The method of claim 7, wherein the donor is not the subject to be treated, regardless of HLA matched status.

10. The method of claim 7, wherein the cells in CPCM are then fractionated by density gradient centrifugation.

11. The method of claim 7, wherein the cells in CPCM are then fractionated by fluorescence-activated cell sorting, magnetic cell beads or other methods known to the art.

12. The method of claim 7, wherein the cells in CPCM are then expanded ex vivo before implantation.

13. The method of claim 7, wherein the cells in CPCM are then cryogenically stored before implantation.

14. The method of claim 7, wherein the cells in CPCM are then included in any suitable formulation or preparation known to the art.

15. The method of claim 7, wherein the cells in CPCM are given in portions that are, but not exclusively limited to, 1×10⁴ to 1×10⁸ cells per treatment.

16. The method of claim 7, wherein the cells in CPCM are being given by different routes of injection, perfusion etc. chosen by medical practitioner familiar with the art.

17. The method of claim 7, wherein the CPCM cells can be used to treat any organs and tissues in need thereof. Non limiting examples of tissues include bone, cartilage, striated, skeletal and cardiac muscle. Non-limiting examples of organs that can be treated by CPCM in include heart, liver, brain, pancreas, kidney, intestine, lung, eye, bladder, spinal cord and skin.
